(19) United States
(12) Patent Application Publication
(10) Pub. No.: US 2016/0199281 A1
(43) Pub. Date: Jul. 14, 2016

(54) HAIR TREATMENT AGENTS CONTAINING OLIGOPEPTIDES AND HYALURONIC ACID ESTERS

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/076,875

(22) Filed: Mar. 22, 2016

Related U.S. Application Data
(63) Continuation of application No. PCT/DE2014/200502, filed on Sep. 24, 2014.

(30) Foreign Application Priority Data
Oct. 2, 2013 (DE) 10 2013 220 061.7

(51) Int. Cl. A61K 8/64; A61K 8/73; A61K 8/84; A61Q 5/00; A61K 8/81

(52) U.S. Cl. CPC A61K 8/64; A61Q 5/002; A61K 8/8135; A61K 8/84; A61K 8/735; A61K 8/817; A61K 2800/591

(57) ABSTRACT

Hair treatment agents include, with respect to the weight thereof, 0.0001-10 wt % of at least one oligopeptide that has at least one amino acid sequence Glu-Glu-Glu (formula (I)), wherein the amino group can be free or protonated and the carboxyl groups can be free or deprotonated, and 0.0001-10 wt % of at least one ester of hyaluronic acid with lipoic acid, formic acid, or mixtures thereof. With such agents, hyaluronic acid esters and certain oligopeptides intensify each other in the positive effect thereof on the hair and the hair follicles.

### FIELD OF THE INVENTION

The present invention generally relates to hair treatment agents, particularly so-called conditioners, having an active-ingredient combination for gently and effectively caring for hair.

### BACKGROUND OF THE INVENTION

The importance of and need for care products having a long-lasting effect are increasing, not least because of the heavy stress on the hair resulting, for example, from dyeing or permanent waves, environmental stresses and from cleaning of the hair with shampoos. Such care agents influence the natural structure and the properties of the hair. Thus, following treatments using beneficial care products, the wet and dry combability of the hair, the hold, and the body of the hair can be optimized or the hair can be protected from increased split ends, for example.

Therefore, it has long been a common technique to subject the hair to a special aftertreatment. In such an aftertreatment, the hair is treated with specific active ingredients, for example quaternary ammonium salts or special polymers, typically in the form of a conditioning agent. By means of this treatment, the combability, the hold, and the body of the hair are improved and the rate of split ends is reduced, depending on the formulation.

The use of specific oligopeptides to care for keratin fibers is known, for example, from WO2010/026010 A1. There, in particular oligopeptides having the sequence Tyr-Glu-Glu-Glu-Ile-Arg-Val-Leu are disclosed as especially suitable for hair care.

Hyaluronic acid and derivatives thereof are favored ingredients in skin cosmetics. They are also increasingly used in hair cosmetics. WO2009/080220 A1 discloses polysaccharides esterified at the hydroxyl function and having glucosamine or galactosamine residues, which are selected, for example, from hyaluronic acid esterified with lipoic acid. These substances have film-forming properties, which strengthen the hair follicles and protect the hair against split ends and external effects of free radicals. In series of tests, improved softness, lightness, and curl retention and a reduction in fragility were exhibited.

There is still a need to provide active ingredients or active-ingredient combinations for cosmetic agents having good care properties and good biodegradability. Particularly in dye- and/or electrolyte-including formulations, there is a need for additional care-providing active ingredients that can be incorporated into known formulations without problems and that are not weakened in the effect thereof there because of incompatibility with other ingredients.

Furthermore, other desirable features and characteristics of the present invention will become apparent from the subsequent detailed description of the invention and the appended claims, taken in conjunction with this background of the invention.

### BRIEF SUMMARY OF THE INVENTION

A hair treatment agent, including, in each case with respect to the weight thereof, 0.0001 to 10 wt % of at least one oligopeptide that comprises at least one amino acid sequence Glu-Glu-Glu

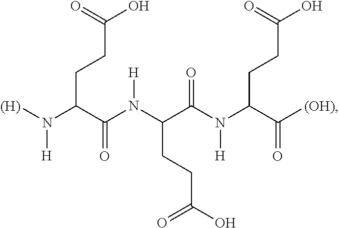

wherein the amino group can be free or protonated and the carboxyl groups can be free or deprotonated. The hair treatment agent also includes 0.0001 to 10 wt % of at least one ester of hyaluronic acid with lipoic acid, formic acid, or mixtures thereof.

A method for improving at least one of the following properties: tensile strength of keratin fibers, particularly human hair; stabilization of the moisture balance of keratin fibers, particularly human hair; combability of keratin fibers, particularly human hair; slowing of the aging process of keratin fibers, particularly human hair; ability of keratin fibers, particularly human hair, to be restructured during and after the permanent wave process; reduction in the elasticity decrease of keratin fibers, particularly human hair, in the event of damage caused by atmospheric influences, includes applying to the keratin fibers a hair treatment agent including, in each case with respect to the weight thereof, 0.0001 to 10 wt % of at least one oligopeptide that includes at least one amino acid sequence Glu-Glu-Glu

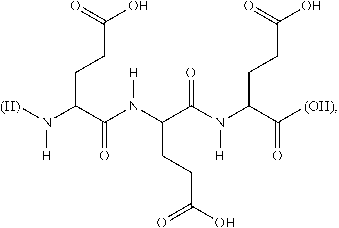

wherein the amino group can be free or protonated and the carboxyl groups can be free or deprotonated, and 0.0001 to 10 wt % of at least one ester of hyaluronic acid with lipoic acid, formic acid, or mixtures thereof. The agent is left there for 5 seconds to 10 minutes, and is then rinsed out.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that hyaluronic acid esters and certain oligopeptides intensify each other in the positive effect thereof on the hair and the hair follicles.

The present invention first relates to hair treatment agents, including—in each case with respect to the weight thereof—
a) 0.0001 to 10 wt % of at least one oligopeptide that comprises at least one amino acid sequence Glu-Glu-Glu

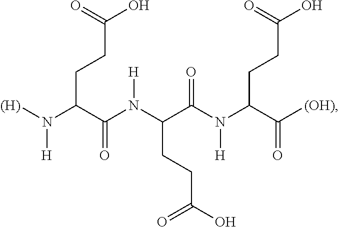

wherein the amino group can be free or protonated and the carboxyl groups can be free or deprotonated
b) 0.0001 to 10 wt % of at least one hyaluronic acid ester with lipoic acid, formic acid, or mixtures thereof.

In this formula as in all formulas below, the bracketed hydrogen atom of the amino group and the bracketed hydroxy group of the acid function mean that the groups in question can be present as such (then the oligopeptide is an oligopeptide having the particular number of amino acids as shown (in formula 3 above)) or that the amino acid sequence is present in an oligopeptide that also comprises further amino acids—depending on where the further amino acid(s) is/are bonded, the bracketed constituents of the formula above are replaced with the further amino acid residue(s).

Within the meaning of the present invention, hair treatment agents are, for example, hair shampoos, hair conditioners, conditioning shampoos, hair sprays, hair conditioning agents, hair treatments, hair packs, hair tonics, permanent-wave fixatives, hair dyeing shampoos, hair dyes, hair setting lotions, hair styling agents, hair styling preparations, blow-dry lotions, mousses, hair gels, hair waxes, or combinations thereof. In light of the fact that men often shy away from the use of several different agents and/or from several usage steps, agents according to the invention are preferably those agents that men use anyway. Therefore, preferred agents according to the invention are shampoos, conditioning media, or hair tonics.

The hair treatment agents according to the invention include, with respect to the weight thereof, 0.0001 to 10 wt % of at least one oligopeptide that comprises at least one amino acid sequence Glu-Glu-Glu, i.e., at least three consecutive glutamic acids.

Within the meaning of the present application, oligopeptides are condensation products of amino acids, which condensation products are linked by peptide bonds in the manner of acid amide and comprise at least 3 and at most 25 amino acids.

In hair treatment agents preferred according to the invention, the oligopeptide comprises 5 to 15 amino acids, preferably 6 to 13 amino acids, especially preferably 7 to 12 amino acids, and particularly 8, 9, or 10 amino acids.

The molar mass of the oligopeptide included in the agents according to the invention can vary, depending on whether further amino acids are bonded to the sequence Glu-Glu-Glu and depending on the type of said amino acids. Hair treatment agents preferred according to the invention are characterized in that the oligopeptide has a molar mass of 650 to 3000 Da, preferably of 750 to 2500 Da, especially preferably of 850 to 2000 Da, and particularly of 1000 to 1600 Da.

In summary, hair treatment agents preferred according to the invention are characterized in that the oligopeptide comprises 5 to 15 amino acids, preferably 6 to 13 amino acids, especially preferably 7 to 12 amino acids, and particularly 8, 9, or 10 amino acids and has a molar mass of 650 to 3000 Da, preferably of 750 to 2500 Da, especially preferably of 850 to 2000 Da, and particularly of 1000 to 1600 Da.

As can be seen from the preferred number of amino acids in the oligopeptides and the preferred molar mass range, oligopeptides that do not consist solely of the three glutamic acids but rather comprise further amino acids bonded to this sequence are preferably used. Said further amino acids are preferably selected from certain amino acids, while certain other representatives are less preferred according to the invention.

Thus, it is preferred if the oligopeptides used in the agents according to the invention do not include methionine.

It is more highly preferred if the oligopeptides used in the agents according to the invention do not include cysteine or cystine.

It is more highly preferred if the oligopeptides used in the agents according to the invention do not include aspartic acid and/or asparagine.

It is more highly preferred if the oligopeptides used in the agents according to the invention do not include serine and/or threonine.

In contrast, it is preferred if the oligopeptides used in the agents according to the invention include tyrosine.

It is more highly preferred if the oligopeptides used in the agents according to the invention include leucine.

It is more highly preferred if the oligopeptides used in the agents according to the invention include isoleucine.

It is more highly preferred if the oligopeptides used in the agents according to the invention include arginine.

It is more highly preferred if the oligopeptides used in the agents according to the invention include valine.

Especially preferred oligopeptides, or rather amino acid sequences included in the preferred oligopeptides, are described below:

An especially preferred oligopeptide additionally includes tyrosine that is preferably bonded to the Glu-Glu-Glu sequence by means of the acid function of tyrosine. Therefore, hair treatment agents preferred according to the invention are characterized in that the oligopeptide included therein comprises at least one amino acid sequence Tyr-Glu-Glu-Glu, wherein the amino group can be free or protonated and the carboxyl groups can be free or deprotonated.

A further especially preferred oligopeptide additionally includes isoleucine that is preferably bonded to the Glu-Glu-Glu sequence by means of the amino function of isoleucine. Therefore, hair treatment agents preferred according to the invention are characterized in that the oligopeptide included therein comprises at least one amino acid sequence Glu-Glu-Glu-Ile, wherein the amino group can be free or protonated and the carboxyl groups can be free or deprotonated.

Oligopeptides that comprise both amino acids mentioned above (tyrosine and isoleucine) are preferred according to the invention, Especially preferred are hair treatment agents according to the invention in the case of which the oligopeptide included therein comprises at least one amino acid sequence Tyr-Glu-Glu-Glu-Ile, wherein the amino group can be free or protonated and the carboxyl groups can be free or deprotonated.

More highly preferred oligopeptides additionally include arginine that is preferably bonded to isoleucine.

Preferred hair treatment agents are characterized in that the oligopeptide included therein comprises at least one amino acid sequence Tyr-Glu-Glu-Glu-Ile-Arg, wherein the amino groups can be free or protonated and the carboxyl groups can be free or deprotonated.

Even more highly preferred oligopeptides additionally include valine that is preferably bonded to the arginine. Therefore, hair treatment agents more highly preferred according to the invention are characterized in that the oligopeptide included therein comprises at least one amino acid sequence Tyr-Glu-Glu-Glu-Ile-Arg-Val, wherein the amino groups can be free or protonated and the carboxyl groups can be free or deprotonated.

Even more highly preferred oligopeptides additionally include leucine that is preferably bonded to the valine. Hair treatment agents more highly preferred according to the invention are characterized in that the oligopeptide included therein comprises at least one amino acid sequence Tyr-Glu-Glu-Glu-Ile-Arg-Val-Leu, wherein the amino groups can be free or protonated and the carboxyl groups can be free or deprotonated.

Particularly preferred oligopeptides additionally include leucine that is preferably bonded to the tyrosine. Hair treatment agents more highly preferred according to the invention are characterized in that the oligopeptide included therein comprises at least one amino acid sequence Leu-Tyr-Glu-Glu-Glu-Ile-Arg-Val-Leu, wherein the amino groups can be free or protonated and the carboxyl groups can be free or deprotonated.

Very especially preferably, agents according to the invention include at least two oligopeptides that satisfy the criteria mentioned above but differ from each other, Thus, for example, hair treatment agents that include at least two oligopeptides A and B that differ from each other and that both include the amino acid sequence Glu-Glu-Glu are preferred.

Such oligopeptides A and B that differ from each other correspond to each other in that they bear three consecutive Glu amino acids in the amino acid sequence thereof, but have differences in the amino acids bonded before or after said three consecutive Glu amino acids. Preferred are peptides that differ from each other and that have partial correspondence, which can be quite larger than in the three amino acids mentioned above.

Thus, more highly preferred hair treatment agents are characterized in that they include at least two oligopeptides A and B that differ from each other and that both include the amino acid sequence Glu-Glu-Glu-Ile.

Likewise preferred are hair treatment agents that include at least two oligopeptides A and B that differ from each other and that both include the amino acid sequence Tyr-Glu-Glu-Glu.

Even more highly preferred hair treatment agents are characterized in that they include at least two oligopeptides A and B that differ from each other and that both include the amino acid sequence Glu-Glu-Glu-Ile-Arg.

Likewise even more highly preferred hair treatment agents include at least two oligopeptides A and B that differ from each other and that both include the amino acid sequence Tyr-Glu-Glu-Glu-Ile.

Very especially preferred hair treatment agents are characterized in that they include at least two oligopeptides A and B that differ from each other and that both include the amino acid sequence Glu-Glu-Glu-Ile-Arg.

Likewise very especially preferred hair treatment agents include at least two oligopeptides A and B that differ from each other and that both include the amino acid sequence Tyr-Glu-Glu-Glu-Ile-Arg.

There is preferably an even larger structural correspondence in the oligopeptides. Thus, hair treatment agents that include at least two oligopeptides A and B that differ from each other and that both include the amino acid sequence Glu-Glu-Glu-Ile-Arg-Val are additional preferred embodiments of the present invention.

Likewise preferred embodiments are hair treatment agents that include at least two oligopeptides A and B that differ from each other and that both include the amino acid sequence Tyr-Glu-Glu-Glu-Ile-Arg-Val.

Even more highly preferred hair treatment agents according to the invention include at least two oligopeptides A and B that differ from each other and that both include the amino acid sequence Glu-Glu-Glu-Ile-Arg-Val-Leu.

Likewise even more highly preferred hair treatment agents include at least two oligopeptides A and B that differ from each other and that both include the amino acid sequence Tyr-Glu-Glu-Glu-Ile-Arg-Val-Leu.

Particularly preferred hair treatment agents are characterized in that they include at least two oligopeptides A and B that differ from each other, the oligopeptide A comprising the amino acid sequence Leu-Tyr-Glu-Glu-Glu-Ile-Arg-Val-Leu, wherein the amino groups can be free or protonated and the carboxyl groups can be free or deprotonated, and the oligopeptide B comprising the amino acid sequence Tyr-Glu-Glu-Glu-Ile-Arg-Val-Leu, wherein the amino groups can be free or protonated and the carboxyl groups can be free or deprotonated.

Very especially preferred agents of this latter embodiment include, with respect to the weight of the agent, 0.00001 to 1 wt % of oligopeptide A and 0.00001 to 1 wt % of oligopeptide B.

More highly preferred agents of this latter embodiment include, with respect to the weight of the agent, 0.00005 to 0.1 wt % of oligopeptide A and 0.00005 to 0.1 wt % of oligopeptide B.

Even more highly preferred agents of this latter embodiment include, with respect to the weight of the agent, 0.0001 to 0.01 wt % of oligopeptide A and 0.0001 to 0.001 wt % of oligopeptide B.

It has been found that the use of the oligopeptides mentioned above give the hair treatment agents according to the invention excellent properties. The agents according to the invention give the hair treated therewith greater elasticity, which is exhibited in higher tensile strengths of the keratin fibers and in a reduction of the elasticity decrease, e.g., in the case of damage caused by atmospheric influences. In particular, the especially preferred oligopeptides also stabilize the moisture balance of the keratin fibers, so that combability is improved and the aging process is slowed.

A further advantage of the compositions according to the invention is that the ability of keratin fibers treated therewith to be shaped and restructured is improved.

The oligopeptides used in the context of the present invention that satisfy the conditions mentioned above can advantageously be obtained from keratin materials. It is preferred according to the invention that said oligopeptides are used in high fractions with respect to the total keratin peptide content of the agents.

It is very especially preferred that a fraction of all keratin peptides included in the agent that is as high as possible satisfies the conditions mentioned above.

Preferred hair treatment agents according to the invention are characterized in that at least 0.1 wt %, preferably at least 0.5 wt %, especially preferably at least 1 wt %, more preferably at least 2.5 wt %, even more preferably at least 5 wt %, and particularly at least 10 wt %, of all keratin peptides included in the agent comprise the amino acid sequence Glu-Glu-Glu.

More highly preferred hair treatment agents according to the invention are characterized in that at least 0.1 wt %, preferably at least 0.5 wt %, especially preferably at least 1 wt %, more preferably at least 2.5 wt %, even more preferably at least 5 wt %, and particularly at least 10 wt %, of all keratin peptides included in the agent comprise the amino acid sequence Glu-Glu-Glu-Ile.

Even more highly preferred hair treatment agents according to the invention are characterized in that at least 0.1 wt %, preferably at least 0.5 wt %, especially preferably at least 1 wt %, more preferably at least 2.5 wt %, even more preferably at least 5 wt %, and particularly at least 10 wt %, of all keratin peptides included in the agent comprise the amino acid sequence Tyr-Glu-Glu-Glu.

Especially preferred hair treatment agents according to the invention are characterized in that at least 0.1 wt %, preferably at least 0.5 wt %, especially preferably at least 1 wt %, more preferably at least 2.5 wt %, even more preferably at least 5 wt %, and particularly at least 10 wt %, of all keratin peptides included in the agent comprise the amino acid sequence Tyr-Glu-Glu-Glu-Ile.

Very especially preferred hair treatment agents according to the invention are characterized in that at least 0.1 wt %, preferably at least 0.5 wt %, especially preferably at least 1 wt %, more preferably at least 2.5 wt %, even more preferably at least 5 wt %, and particularly at least 10 wt %, of all keratin peptides included in the agent comprise the amino acid sequence Tyr-Glu-Glu-Glu-Ile-Arg.

Even more highly preferred hair treatment agents according to the invention are characterized in that at least 0.1 wt %, preferably at least 0.5 wt %, especially preferably at least 1 wt %, more preferably at least 2.5 wt %, even more preferably at least 5 wt %, and particularly at least 10 wt %, of all keratin peptides included in the agent comprise the amino acid sequence Tyr-Glu-Glu-Glu-Ile-Arg-Val.

Particularly preferred hair treatment agents according to the invention are characterized in that at least 0.1 wt %, preferably at least 0.5 wt %, especially preferably at least 1 wt %, more preferably at least 2.5 wt %, even more preferably at least 5 wt %, and particularly at least 10 wt %, of all keratin peptides included in the agent comprise the amino acid sequence Tyr-Glu-Glu-Glu-Ile-Arg-Val-Leu.

The conditions mentioned above relate to the total content of peptides originating from keratin materials in the agents according to the invention. Of course, in addition to the oligopeptides of keratin origin, further peptides and/or protein hydrolysates, for example from other native sources, can be used. For example, the additional use of wheat protein hydrolysates is preferred (see further below).

The hair treatment agents according to the invention include, with respect to the weight of said hair treatment agents, 0.0001 to 10 wt % of at least one ester of hyaluronic acid with lipoic acid, formic acid, or mixtures thereof as a second essential ingredient.

Accordingly, the agents according to the invention can include esters of hyaluronic acid with lipoic acid, esters of hyaluronic acid with formic acid, or mixed esters of hyaluronic acid with lipoic acid and formic acid. The latter are especially preferred with regard to the effectiveness thereof in the combination according to the invention.

Hair treatment agents preferred according to the invention are characterized in that they include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid, formic acid, or mixtures thereof of formula (I)

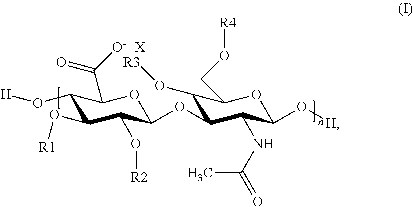

in which
X is selected from H, Na, and K,
n stands for numbers from 100 to 10000, and
R1, R2, R3, and R4 are selected independently of each other from —H, —C(O)—H, and

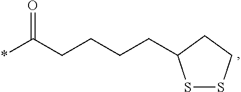 [SMILES: *C(=O)CCCCC1CCSS1]

with the stipulation at least one of the residues R1, R2, R3, and R4 is different from —H.

The hyaluronic acid has 4 hydroxyl groups that can be esterified, and therefore, with respect to the individual recurring unit, the following hyaluronic esters can be used according to the invention:
hyaluronic acid esters in which one OH group is esterified with lipoic acid,
hyaluronic acid esters in which two OH groups are esterified with lipoic acid,
hyaluronic acid esters in which three OH groups are esterified with lipoic acid,
hyaluronic acid esters in which four OH groups are esterified with lipoic acid,
hyaluronic acid esters in which one OH group is esterified with formic acid,
hyaluronic acid esters in which two OH groups are esterified with formic acid,
hyaluronic acid esters in which three OH groups are esterified with formic acid,
hyaluronic acid esters in which four OH groups are esterified with formic acid,
hyaluronic acid esters in which one OH group is esterified with lipoic acid and one OH group is esterified with formic acid,
hyaluronic acid esters in which one OH group is esterified with lipoic acid and two OH groups are esterified with formic acid,
hyaluronic acid esters in which one OH group is esterified with lipoic acid and three OH groups are esterified with formic acid,
hyaluronic acid esters in which two OH groups are esterified with lipoic acid and one OH group is esterified with formic acid,
hyaluronic acid esters in which two OH groups are esterified with lipoic acid and two OH groups are esterified with formic acid,
hyaluronic acid esters in which three OH groups are esterified with lipoic acid and one OH group is esterified with formic acid.

The esterification occurs statistically, and therefore individual monomer units can remain completely unesterified. It is preferred according to the invention if a certain portion of the OH groups that can be esterified is esterified with lipoic acid, regardless of how said OH groups are distributed on the hyaluronic acid molecule.

Hair treatment agents preferred according to the invention are characterized in that the degree of esterification of the OH groups with lipoic acid is 0.1 to 0.5 mol/mol, preferably 0.125 to 0.45 mol/mol, more preferably 0.2 to 0.4 mol/mol, even more preferably 0.25 to 0.35 mol/mol, and particularly 0.27 to 0.33 mol/mol. In other words, on 25 repeat units (having 100 OH groups), extremely preferably 27 to 33 OH groups are esterified with lipoic acid.

It is more highly preferred according to the invention if a certain portion of the OH groups that can be esterified is esterified with formic acid, likewise regardless of how said OH groups are distributed on the hyaluronic acid molecule.

Hair treatment agents preferred according to the invention are characterized in that the degree of esterification of the OH groups with formic acid is 0.001 to 0.5 mol/mol, preferably 0.002 to 0.4 mol/mol, more preferably 0.005 to 0.3 mol/mol, even more preferably 0.0075 to 0.25 mol/mol, and particularly 0.01 to 0.2 mol/mol. In other words, on 25 repeat units (having 100 OH groups), extremely preferably 1 to 20 OH groups are esterified with formic acid.

It is extremely preferred that both conditions are satisfied simultaneously. Especially preferred hair treatment agents according to the invention are characterized in that the degree of esterification of the OH groups with lipoic acid is 0.1 to 0.5 mol/mol, preferably 0.125 to 0.45 mol/mol, more preferably 0.2 to 0.4 mol/mol, even more preferably 0.25 to 0.35 mol/mol, and particularly 0.27 to 0.33 mol/mol, and the degree of esterification of the OH groups with formic acid is 0.001 to 0.5 mol/mol, preferably 0.002 to 0.4 mol/mol, more preferably 0.005 to 0.3 mol/mol, even more preferably 0.0075 to 0.25 mol/mol, and particularly 0.01 to 0.2 mol/mol. In other words, on 25 repeat units (having 100 OH groups), extremely preferably 27 to 33 OH groups are esterified with lipoic acid and 1 to 20 OH groups are esterified with formic acid.

As already mentioned, the use of hyaluronic acid esters in the case of which extremely preferably 27 to 33 OH groups are esterified with lipoic acid on 25 repeat units (having 100 OH groups) is preferred. Accordingly, preferred hair treatment agents include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid, said at least one ester comprising at least one recurring unit of formula (II)

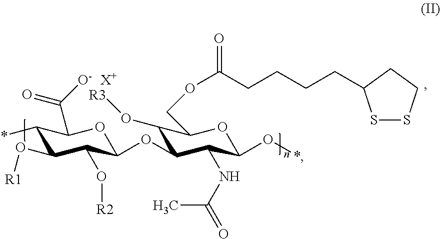

in which
X is selected from H, Na, and K,
n stands for numbers from 1 to 10000, and
R1, R2, and R3 are selected independently of each other from —H, —C(O)—H, and

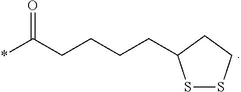 [SMILES: *C(=O)CCCCC1CCSS1]

More highly preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid of formula (IIa)

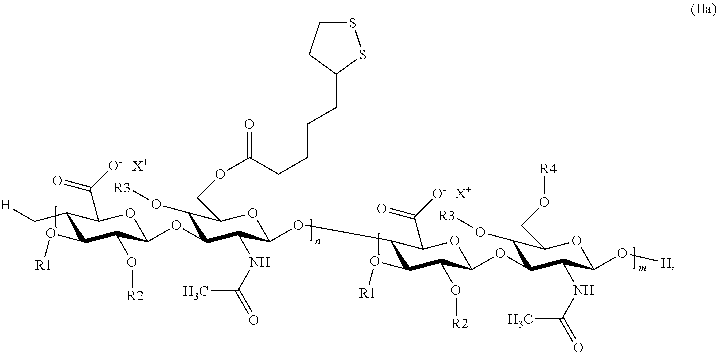

in which
X is selected from H, Na, and K,
n and m stand for numbers from 100 to 10000, and
R1, R2, R3, and R4 are selected independently of each other from —H, —C(O)—H, and

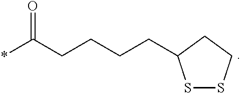 [SMILES: *C(=O)CCCCC1CCSS1]

In formula (IIa), the recurring units having the indices n and m do not necessarily have to be bonded to each other as a block. Rather, they can be statistically distributed over the entire molecule.

Even more highly preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid of formula (IIb)

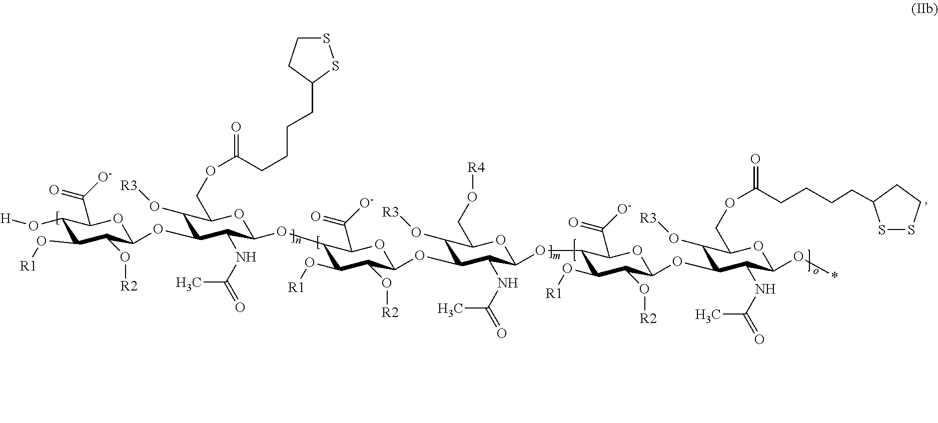

in which
X is selected from H, Na, and K,
n, m, and o stand for numbers from 100 to 10000, and
R1, R2, R3, and R4 are selected independently of each other from —H, —C(O)—H, and

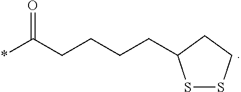 [SMILES: *C(=O)CCCCC1CCSS1]

Likewise here in formula (IIb), the recurring units having the indices n, m, and o do not necessarily have to be bonded to each other as a block. Rather, they can be statistically distributed over the entire molecule.

Likewise preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid, said at least one ester comprising at least one recurring unit of formula (III)

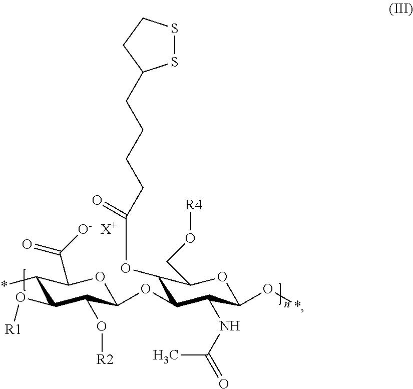

in which
X is selected from H, Na, and K,
n stands for numbers from 1 to 10000, and
R1, R2, and R4 are selected independently of each other from —H, —C(O)—H, and

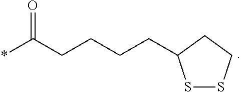 [SMILES: *C(=O)CCCCC1CCSS1]

More highly preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid of formula (IIIa)

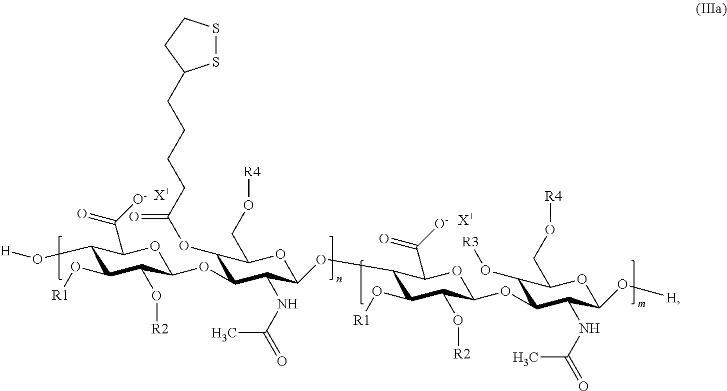

in which
X is selected from H, Na, and K,
n and m stand for numbers from 100 to 10000, and
R1, R2, R3, and R4 are selected independently of each other from —H, —C(O)—H, and

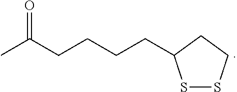

In formula (IIIa), the recurring units having the indices n and m do not necessarily have to be bonded to each other as a block. Rather, they can be statistically distributed over the entire molecule.

Even more highly preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid of formula (IIIb)

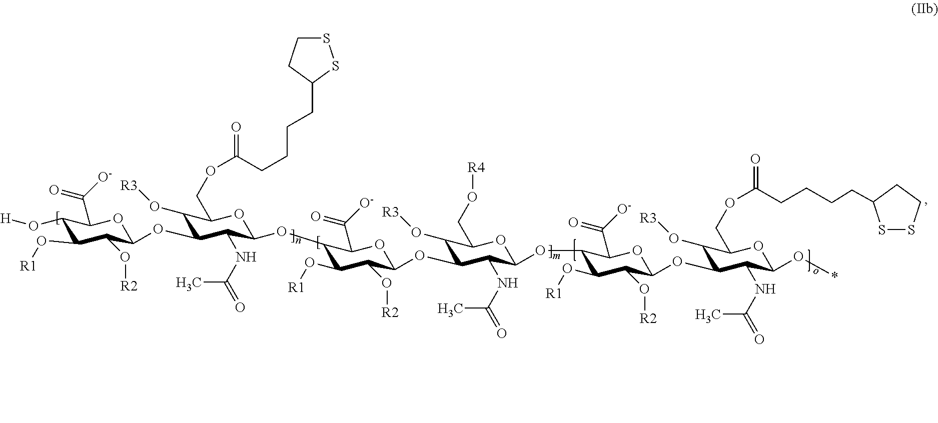

in which
X is selected from H, Na, and K,
n, m, and o stand for numbers from 100 to 10000, and
R1, R2, R3, and R4 are selected independently of each other from —H, —C(O)—H, and

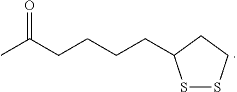

Likewise here in formula (IIIb), the recurring units having the indices n, m, and o do not necessarily have to be bonded to each other as a block. Rather, they can be statistically distributed over the entire molecule.

Likewise preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid, said at least one ester comprising at least one recurring unit of formula (IV)

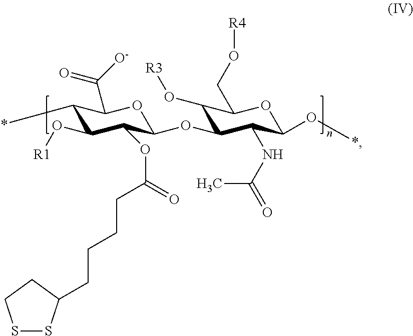

in which
X is selected from H, Na, and K,
n stands for numbers from 1 to 10000, and
R1, R3, and R4 are selected independently of each other from —H, —C(O)—H, and

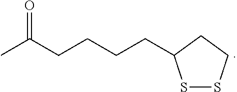

More highly preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid of formula (IVa)

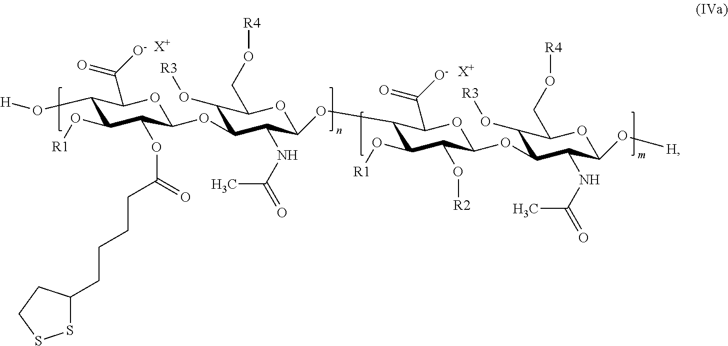

in which
X is selected from H, Na, and K,
n and m stand for numbers from 100 to 10000, and
R1, R2, R3, and R4 are selected independently of each other from —H, —C(O)—H, and

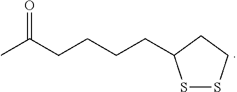

In formula (IVa), the recurring units having the indices n and m do not necessarily have to be bonded to each other as a block. Rather, they can be statistically distributed over the entire molecule.

Even more highly preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid of formula (IVb)

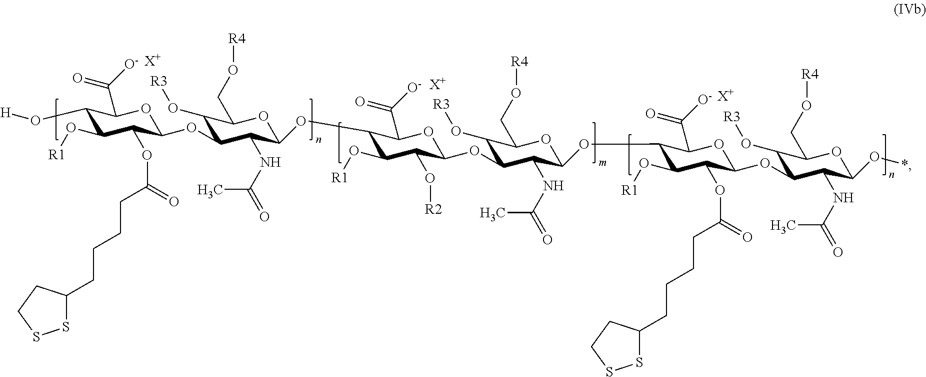

in which
X is selected from H, Na, and K,
n, m, and o stand for numbers from 100 to 10000, and
R1, R2, R3, and R4 are selected independently of each other from —H, —C(O)—H, and

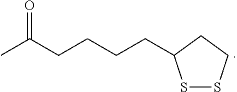

Likewise here in formula (IVb), the recurring units having the indices n, m, and o do not necessarily have to be bonded to each other as a block. Rather, they can be statistically distributed over the entire molecule.

Likewise preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid, said at least one ester comprising at least one recurring unit of formula (V)

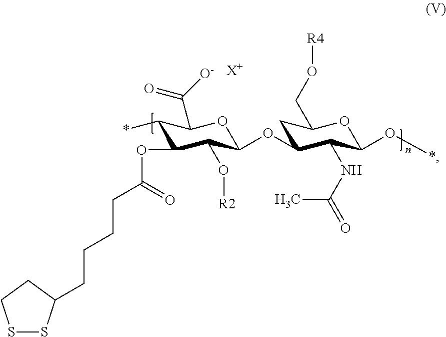

in which
X is selected from H, Na, and K,
n stands for numbers from 1 to 10000, and
R2, R3, and R4 are selected independently of each other from —H, —C(O)—H, and

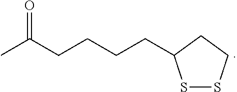

More highly preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid of formula (Va)

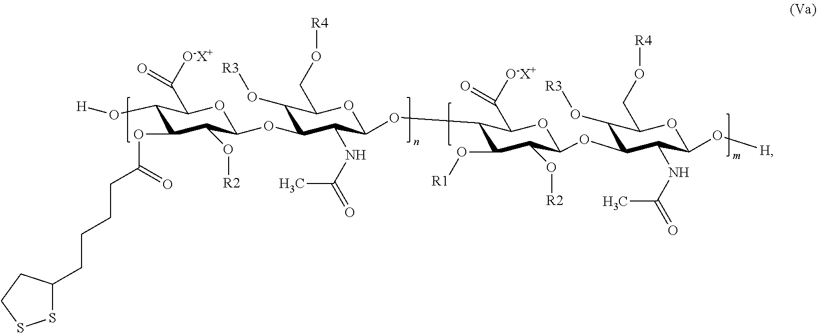

in which
X is selected from H, Na, and K,
n and m stand for numbers from 100 to 10000, and
R1, R2, R3, and R4 are selected independently of each other from —H, —C(O)—H, and

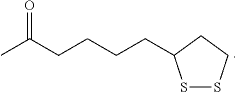

In formula (Va), the recurring units having the indices n and m do not necessarily have to be bonded to each other as a block. Rather, they can be statistically distributed over the entire molecule.

Even more highly preferred hair treatment agents according to the invention include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with lipoic acid of formula (Vb)

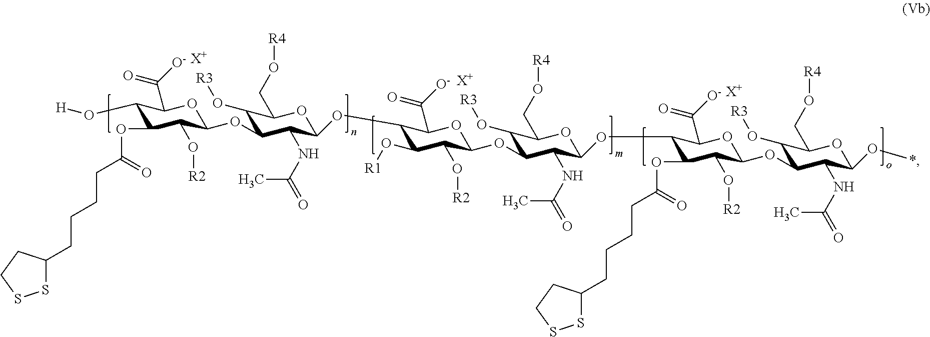

in which
X is selected from H, Na, and K,
n, m, and o stand for numbers from 100 to 10000, and
R1, R2, R3, and R4 are selected independently of each other from —H, —C(O)—H, and

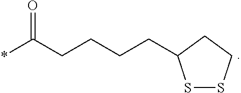 [SMILES: *C(=O)CCCCC1CCSS1]

Likewise here in formula (Vb), the recurring units having the indices n, m, and o do not necessarily have to be bonded to each other as a block. Rather, they can be statistically distributed over the entire molecule.

As already mentioned, the use of hyaluronic acid esters in the case of which extremely preferably 1 to 20 OH groups are esterified with formic acid on 25 repeat units (having 100 OH groups) is preferred. Accordingly, preferred hair treatment agents include 0.0005 to 7.5 wt %, preferably 0.001 to 6 wt %, more preferably 0.005 to 5 wt %, more preferably 0.01 to 4 wt %, and particularly 0.1 to 3 wt %, of at least one ester of hyaluronic acid with formic acid, said at least one ester comprising at least one recurring unit of formula (VI)

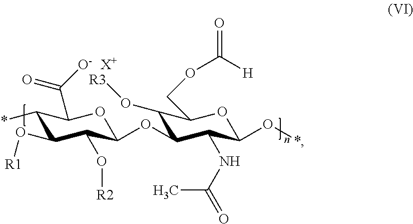

in which
X is selected from H, Na, and K,
n stands for numbers from 100 to 10000, and
R1, R2, and R3 are selected independently of each other from —H, —C(O)—H, and

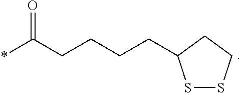 [SMILES: *C(=O)CCCCC1CCSS1]

The agents according to the invention can include further active ingredients and auxiliary substances. These are described below.

The agents according to the invention preferably additionally include at least one emulsifier or one surfactant, wherein surface-active substances are referred to as surfactants or as emulsifiers depending on the field of application and are selected from anionic, cationic, zwitterionic, ampholytic, and nonionic surfactants and emulsifiers.

Hair treatment agents preferred according to the invention are characterized in that they include, with respect to the weight thereof, 0.5 to 70 wt %, preferably 1 to 60 wt %, and particularly 5 to 25 wt %, of anionic and/or nonionic and/or cationic and/or amphoteric surfactant(s).

All anionic surface-active substances suitable for use on the human body are suitable as anionic surfactants and emulsifiers for the compositions according to the invention. Said anionic surface-active substances are characterized by an anionic group that provides water solubility, such as a carboxylate, sulfate, sulfonate, or phosphate group, and a lipophilic alkyl group having approximately 8 to 30 C atoms. In addition, glycol or polyglycol ether groups, ester, ether, and amide groups, and hydroxyl groups can be included in the molecule.

Those surface-active compounds that bear at least one quaternary ammonium group and at least one —COO⁽⁻⁾ or —SO₃⁽⁻⁾ group in the molecule are referred to as zwitterionic surfactants and emulsifiers. Especially suitable zwitterionic surfactants and emulsifiers are the so-called betaines, such as the N-alkyl-N,N-dimethyl ammonium glycinates, for example coco alkyl dimethyl ammonium glycinate, N-acyl-aminopropyl-N,N-dimethyl ammonium glycinates, for example coco acyl aminopropyl dimethyl ammonium glycinate, 2-alkyl-3-carboxymethyl-3-hydroxyethyl imidazolines each having 8 to 18 C atoms in the alkyl or acyl group, and coco acylaminoethyl hydroxyethyl carboxymethyl glycinate. A preferred zwitterionic surfactant is the fatty acid amide derivative known under the INCI name Cocamidopropyl Betaine.

The term “ampholytic surfactants and emulsifiers” refers to those surface-active compounds that include at least one free amino group and at least one —COOH or —SO₃H group in addition to a C₈-C₂₄ alkyl or acyl group and are capable of forming inner salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkytaurines, N-alkylsarcosines, 2-alkylaminopropionic acids, and alkylaminoacetic acids each having approximately 8 to 24 C atoms in the alkyl group. Especially preferred ampholytic surfactants are N-cocoalkylaminopropionate, cocoacylaminoethylaminopropionate, and C₁₂-C₁₈ acylsarco sine.

Nonionic surfactants and emulsifiers include, e.g., a polyol group, a polyalkylene glycol ether group, or a combination of polyol group and polyglycol ether group as a hydrophilic group.

Very especially preferred are agents according to the invention that additionally include fatty alcohol(s) and/or fatty alcohol alkoxylate(s), preferably C₁₂₋₂₂ fatty alcohol(s) and/or C₁₂₋₂₂ fatty alcohol ethoxylate(s) having 10 to 30 EO units, especially preferably C₁₆₋₁₈ fatty alcohol(s) and/or C₁₆₋₁₈ fatty alcohol ethoxylate(s) having 12 to 20 EO units, preferably in amounts of 5 to 20 wt %, preferably 7.5 to 17.5 wt %, and particularly 10 to 15 wt %, in each case with respect to the weight of the agent.

In summary, preferred are hair treatment agents according to the invention that include, with respect to the weight thereof, 0.1 to 20 wt %, preferably 0.25 to 17.5 wt %, and particularly 5 to 15 wt %, of anionic surfactant(s), especially preferably fatty alcohol ether sulfates of the formula

H₃C—(CH₂)ₙ—(OCH₂CH₂)ₖ—OSO₃⁻M⁺,

in which n stands for values of 5 to 21, preferably 7 to 19, especially preferably 9 to 17, and particularly 11 to 13, k stands for values of 1, 2, 3, 4, 5, 6, 7, 8, 9, or 10, preferably for 1, 2, or 3, and particularly for 2, and M stands for a cation from the group Na⁺, K⁺, NH₄⁺, ½ Mg²⁺, ½ Zn²⁺, preferably for Na⁺.

As a further optional constituent, the agents according to the invention include 0.01 to 10 wt % of at least one polymer from the group of the cationic and/or amphoteric polymers.

The term “cationic or amphoteric polymers” refers to polymers that comprise, in the main chain and/or side chain, a group that can be “temporarily” or “permanently” cationic. According to the invention, those polymers that have a cationic group regardless of the pH value of the agent are referred to as “permanently cationic”. These are generally polymers that include a quaternary nitrogen atom, for example in the form of an ammonium group. Preferred cationic groups are quaternary ammonium groups. In particular, those polymers in which the quaternary ammonium group is bonded to a polymer main chain constructed of acrylic acid, methacrylic acid, or derivatives thereof by means of a C1-4 hydrocarbon group have proven to be especially suitable.

In addition to or instead of cationic polymerizates, the agents according to the invention can also include amphoteric polymers. These additionally have at least one negatively charged group in the molecule and are also referred to as zwitterionic polymers.

The polymer or polymers are preferably used within narrow amount ranges. Thus, preferred are agents according to the invention that include, with respect to the weight thereof, 0.05 to 7.5 wt %, preferably 0.1 to 5 wt %, especially preferably 0.2 to 3.5 wt %, and particularly 0.25 to 2.5 wt %, of amphoteric polymer(s).

Regardless of whether amphoteric polymers are included in the agents or not, more highly preferred agents according to the invention are characterized in that they include, with respect to the weight thereof, 0.05 to 7.5 wt %, preferably 0.1 to 5 wt %, especially preferably 0.2 to 3.5 wt %, and particularly 0.25 to 2.5 wt %, of cationic polymer(s).

In addition to or instead of the cationic polymers, the agents according to the invention can include amphoteric polymers.

As a further ingredient, the agents according to the invention can especially preferably include one or more amino acids. Amino acids especially preferably usable according to the invention come from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-CySS), L-carnitine, L-citrulline, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the invention include one or more amino acids in narrow amount ranges. Here, hair treatment agents preferred according to the invention are characterized in that they include, with respect to the weight thereof, 0.01 to 5 wt %, preferably 0.02 to 2.5 wt %, especially preferably 0.05 to 1.5 wt %, more preferably 0.075 to 1 wt %, and particularly 0.1 to 0.25 wt %, of amino acid(s), preferably from the group of glycine and/or alanine and/or valine and/or lysine and/or leucine and/or threonine, as a care substance.

Furthermore, the agents according to the invention can include all active ingredients, additives, and auxiliary substances known for such preparations. In many cases, the agents include at least one surfactant, wherein in principle anionic surfactants as well as zwitterionic, ampholytic, nonionic, and cationic surfactants are suitable. However, in many cases, it has proven to be advantageous to select the surfactants from anionic, zwitterionic, or nonionic surfactants. These surfactants were described in detail further above.

In a further preferred embodiment, the agents according to the invention can include emulsifiers (F). Emulsifiers cause the formation of water- or oil-stable adsorption layers at the phase interface, which adsorption layers protect the dispersed drops against coalescence and thus stabilize the emulsion. Therefore, like surfactants, emulsifiers are constructed of a hydrophobic molecule part and a hydrophilic molecule part. Hydrophilic emulsifiers preferably form O/W emulsions and hydrophobic emulsifiers preferably form W/O emulsions. The term “emulsion” refers to a distribution (dispersion), in the form of drops, of a liquid in another liquid achieved by applying energy in order to create stabilizing phase boundaries by means of surfactants. The selection of these emulsifying surfactants or emulsifiers is based on the substances to be dispersed and the outer phase and the fineness of the emulsion.

Finally, the agents according to the invention can also include plant extracts (L).

Said extracts are typically produced by extracting from the entire plant. However, in individual cases, it can also be preferred to produce the extracts exclusively from flowers and/or leaves of the plant.

The silicones are an especially preferred group of ingredients.

Agents especially preferred according to the invention include the silicone(s) preferably in amounts of 0.1 to 10 wt %, preferably 0.25 to 7 wt %, and particularly 0.5 to 5 wt %, in each case with respect to the entire agent.

Preferred silicones are described below.

Especially preferred agents according to the invention are characterized in that they include at least one silicone of formula Si-I

(CH₃)₃Si—[O—Si(CH₃)₂]ₓ—O—Si(CH₃)₃ (Si-I),

in which x stands for a number from 0 to 100, preferably from 0 to 50, more preferably from 0 to 20, and particularly from 0 to 10.

Said silicones are referred to as DIMETHICONE in accordance with the INCI nomenclature.

Preferred silicones that are usable according to the invention have viscosities of 0.2 to 2 mm²s⁻¹ at 20° C., wherein silicones having viscosities of 0.5 to 1 mm²s⁻¹ are especially preferred.

Especially preferred agents according to the invention include one or more amino-functional silicones. Such silicones can be described, e.g., by the formula

M(RₐQ_{b}SiO)_{4-a-b)/2)x}(R_{c}SiO)_{4-c)/2)y}M,

wherein in the formula above R is a hydrocarbon or a hydrocarbon residue having 1 to approximately 6 carbon atoms, Q is a polar residue of the general formula —R′HZ, wherein R¹ is a divalent, bonding group that is bonded to hydrogen and to the residue Z, composed of carbon and hydrogen atoms, carbon, hydrogen, and oxygen atoms, or carbon, hydrogen, and nitrogen atoms, and Z is an organic, amino-functional residue that includes at least one amino-functional group; “a” assumes values in the range from approximately 0 to approximately 2, “b” assumes values in the range from approximately 1 to approximately 3, “a”+“b” is less than or equal to 3, and “c” is a number in the range from approximately 1 to approximately 3, and x is a number in the range from 1 to approximately 2,000, preferably from approximately 3 to approximately 50, and most preferably from approximately 3 to approximately 25, and y is a number in the range from approximately 20 to approximately 10,000, preferably from approximately 125 to approximately 10,000, and most preferably from approximately 150 to approximately 1,000, and M is a suitable silicone terminal group known in the prior art, preferably trimethylsiloxy. Non-restrictive examples of the residues represented by R include alkyl residues, such as methyl, ethyl, propyl, isopropyl, butyl, isobutyl, amyl, isoamyl, hexyl, isohexyl, and the like; alkenyl resides, such as vinyl, halogen vinyl, alkyl vinyl, allyl, halogen allyl, alkyl allyl; cycloalkyl residues, such as cyclobutyl, cyclopentyl, cyclohexyl, and the like; phenyl residues, benzyl residues, halogen hydrocarbon residues, such as 3-chloropropyl, 4-bromobutyl, 3,3,3-trifluoropropyl, chlorocyclohexyl, bromophenyl, chlorophenyl, and the like; and sulfur-including residues, such as mercaptoethyl, mercaptopropyl, mercaptohexyl, mercaptophenyl, and the like; R is preferably an alkyl residue that includes 1 to approximately 6 carbon atoms, and R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, amino-functional residue including at least one functional amino group. A possible formula for Z is NH(CH₂)_{z}NH₂, wherein z is 1 or more. Another possible formula for Z is —NH(CH₂)_{z}(CH₂)_{zz}NH, wherein both z and zz are independently 1 or more, wherein this structure comprises diamino ring structures, such as piperazinyl. Z is most preferably an —NHCH₂CH₂NH₂ residue. Another possible formula for Z is —N(CH₂)_{z}(CH₂)_{zz}NX₂ or —NX₂, wherein each X of X₂ is independently selected from the group comprising hydrogen and alkyl groups having 1 to 12 carbon atoms, and zz is 0.

Q is most preferably a polar, amino-functional residue of the formula —CH₂CH₂CH₂NHCH₂CH₂NH₂. In the formulas, “a” assumes values in the range from approximately 0 to approximately 2, “b” assumes values in the range from approximately 2 to approximately 3, “a”+“b” is less than or equal to 3, and “c” is a number in the range from approximately 1 to approximately 3. The molar ratio of the RₐQ_{b} SiO_{4-a-b)/2} units to the R_{c}SiO_{(4-c)/2} units lies in the range from approximately 1:2 to 1:65, preferably from approximately 1:5 to approximately 1:65, and most preferably from approximately 1:15 to approximately 1:20. If one or more silicones of the formula above are used, then the various variable substituents in the formula above can be different in the various silicone components that are present in the silicone mixture.

Preferred agents according to the invention are characterized in that they include an amino-functional silicone of formula (Si-II)

R′ₐG₃₋ₐ-Si(OSiG₂)ₙ-(OSiG_{b}R′_{2-b})ₘ—O—SiG₃₋ₐ-R′ₐ (Si-II),

wherein:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a stands for a number between 0 and 3, particularly 0;
b stands for a number between 0 and 1, particularly 1,
m and n are numbers, the sum (m+n) of which is between 1 and 2000, preferably between 50 and 150, wherein n preferably assumes values from 0 to 1999 and particularly from 49 to 149 and m preferably assumes values from 1 to 2000, particularly from 1 to 10,
R′ is a monovalent residue selected from
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻
wherein each Q stands for a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
R″ stands for identical or differing residues from the group —H, -phenyl, -benzyl, —CH₂—CH(CH₃)Ph, the C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion, which is preferably selected from chloride, bromide, iodide, or methosulfate.

Especially preferred agents according to the invention are characterized in that they include at least one amino-functional silicone of formula (Si-IIa)

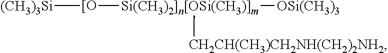

wherein m and n are numbers, the sum (m+n) of which is between 1 and 2000, preferably between 50 and 150, wherein n preferably assumes values from 0 to 1999 and particularly from 49 to 149 and m preferably assumes values from 1 to 2000, particularly from 1 to 10.

These silicones are referred to as Trimethylsilylamodimethicone in accordance with the INCI declaration.

Also especially preferred are agents according to the invention that include an amino-functional silicone of formula (Si-IIb)

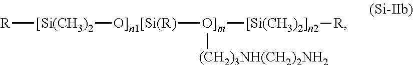

wherein R stands for —OH, —O—CH₃, or a —CH₃ group and m, n1, and n2 are numbers, the sum (m+n1+n2) of which is between 1 and 2000, preferably between 50 and 150, wherein the sum (n1+n2) preferably assumes values from 0 to 1999, particularly from 49 to 149, and m preferably assumes values from 1 to 2000, particularly from 1 to 10.

These silicones are referred to as Amodimethicone in accordance with the INCI declaration.

Regardless of which amino-functional silicones are used, preferred are agents according to the invention that include an amino-functional silicone, the amine value of which lies above 0.25 mEq/g, preferably above 0.3 mEq/g, and particularly above 0.4 mEq/g. The amine value stands for the milliequivalents of amine per gram of the amino-functional silicone. The amine value can be determined by titration and can also be specified in the unit mg KOH/g.

Agents preferred according to the invention are characterized in that they include, with respect to the weight thereof, 0.01 to 10 wt %, preferably 0.1 to 8 wt %, especially preferably 0.25 to 7.5 wt %, and particularly 0.5 to 5 wt %, of amino-functional silicone(s).

The cyclic dimethicones referred to as CYCLOMETHICONE in accordance with INCI can be used with preference according to the invention. Preferred here are agents according to the invention that include at least one silicone of formula Si-III

—[O—Si(CH₃)₂]ₓ— (Si-III),

in which x stands for a number from 3 to 200, preferably from 3 to 10, more preferably from 3 to 7, and particularly 3, 4, 5, or 6.

The silicones described above comprise a backbone that is constructed of —Si—O—Si— units. Of course, said —Si—O—Si— units can also be interrupted by carbon chains. Corresponding molecules can be obtained by means of chain extension reactions and are preferably used in the form of silicone-in-water emulsions.

Agents likewise preferred according to the invention are characterized in that they include at least one silicone of formula Si-IV

R₃Si—[O—SiR₂]ₓ—(CH₂)ₙ—[O—SiR₂]_{y}—O—SiR₃ (Si-IV),

in which R stands for identical or different residues from the group of —H, -phenyl, -benzyl, —CH₂—CH(CH₃)Ph, the C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y each stand for a number from 0 to 200, preferably from 0 to 10, more preferably from 0 to 7, and particularly 0, 1, 2, 3, 4, 5, or 6, and n stands for a number from 0 to 10, preferably from 1 to 8, and particularly for 2, 3, 4, 5, 6.

The silicones are preferably water-soluble. Agents preferred according to the invention are characterized in that they include at least one water-soluble silicone.

For aesthetic reasons, “clear” products are often preferred by consumers. Therefore, hair treatment agents preferred according to the invention are characterized in that they are transparent or translucent. In the context of the present invention, transparent or translucent means a composition that has an NTU value of less than 100. The NTU value (Nephelometric Turbidity Unit; NTU) is a unit for turbidity measurements in liquids that is used in water treatment. It is the unit of a turbidity of a liquid measured by means of a calibrated nephelometer.

The agents according to the invention have advantageous properties and give likewise advantageous properties to the hair treated therewith. Particularly in the treatment of the hair and scalp, advantages that go beyond the advantages of the oligopeptides or hyaluronic acid esters alone were observed. Thus, hair treatment agents according to the invention increase the elasticity of the hair treated therewith and lead to a strengthening of the hair fibers within the structure thereof, which strengthening is reflected, for example, in higher melting temperatures in differential thermal analysis.

An improvement in wet and dry combability and prevention of premature formation of split ends in the treated hair are also exhibited. On the skin and particularly the scalp, the agents according to the invention cause an increase in elasticity and, surprisingly, sebum-regulating effects. The optical impression of “oily” skin or hair is thus avoided or diminished.

The present invention also relates to a method for improving at least one of the following properties:
tensile strength of keratin fibers, particularly human hair;
stabilization of the moisture balance of keratin fibers, particularly human hair;
combability of keratin fibers, particularly human hair;
slowing of the aging process of keratin fibers, particularly human hair;
ability of keratin fibers, particularly human hair, to be restructured during and after the permanent wave process;
reduction in the elasticity decrease of keratin fibers, particularly human hair, in the event of damage caused by atmospheric influences

wherein a hair treatment agent including, in each case with respect to the weight thereof,
a) 0.0001 to 10 wt % of at least one oligopeptide that comprises at least one amino acid sequence Glu-Glu-Glu, wherein the amino group can be free or protonated and the carboxyl groups can be free or deprotonated,
b) 0.0001 to 10 wt % of at least one ester of hyaluronic acid with lipoic acid, formic acid, or mixtures thereof
   is applied to the keratin fibers, left there for 5 seconds to 10 minutes, and then rinsed out.

What has been said in regard to the agents according to the invention applies, mutatis mutandis, to further preferred embodiments of the method according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent, including, in each case with respect to the weight thereof,
a) 0.0001 to 10 wt % of at least one oligopeptide that comprises at least one amino acid sequence Glu-Glu-Glu 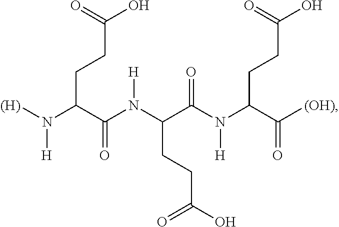
wherein the amino group can be free or protonated and the carboxyl groups can be free or deprotonated, and
b) 0.0001 to 10 wt % of at least one ester of hyaluronic acid with lipoic acid, formic acid, or mixtures thereof.

2. The hair treatment agent according to claim 1, wherein the oligopeptide comprises 5 to 15 amino acids and has a molar mass of 650 to 3000 Da.

3. The hair treatment agent according to claim 1, wherein the oligopeptide comprises Tyr-Glu-Glu-Glu-Ile 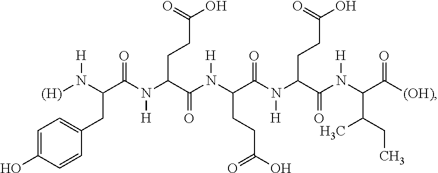
wherein the amino group can be free or protonated and the carboxyl groups can be free or deprotonated.

4. The hair treatment agent according to claim 1, wherein the oligopeptide comprises at least one amino acid sequence Tyr-Glu-Glu-Glu-Ile-Arg-Val-Leu 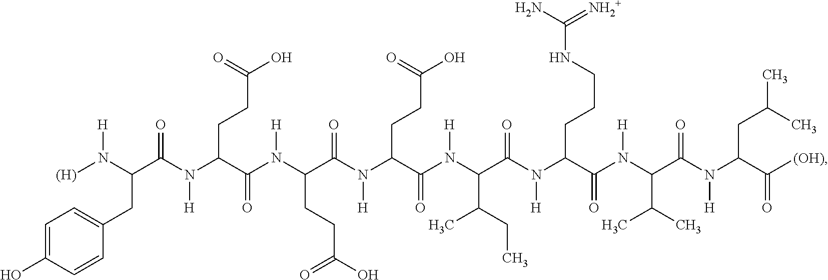
wherein the amino groups can be free or protonated and the carboxyl groups can be free or deprotonated.

5. The hair treatment agent according to claim 1, wherein the hair treatment agent includes 0.0005 to 7.5 wt % of at least one ester of hyaluronic acid with lipoic acid, formic acid, or mixtures thereof of formula (I) 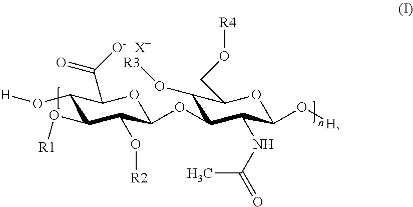
wherein
X is selected from H, Na, and K,
n stands for numbers from 100 to 10000, and
R1, R2, R3, and R4 are selected independently of each other from —H, —C(O)—H, and 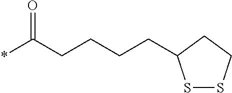 [SMILES: *C(=O)CCCCC1CCSS1]
with the stipulation that at least one of the residues R1, R2, R3, and R4 is different from —H.

6. The hair treatment agent according to claim 1, wherein the hair treatment agent includes 0.0005 to 7.5 wt % of at least one ester of hyaluronic acid with lipoic acid, wherein at least one such ester comprises at least one recurring unit of formula (II) 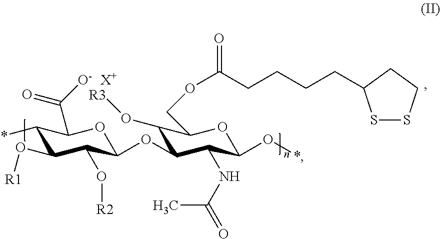
wherein
X is selected from H, Na, and K,
n stands for numbers from 1 to 10000, and
R1, R2, and R3 are selected independently of each other from —H, —C(O)—H, and

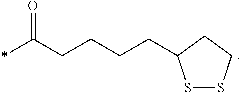 [SMILES: *C(=O)CCCCC1CCSS1]

7. The hair treatment agent according to claim 1, wherein the hair treatment agent includes 0.0005 to 7.5 wt % of at least one ester of hyaluronic acid with formic acid, wherein at least one ester comprises at least one recurring unit of formula (VI) 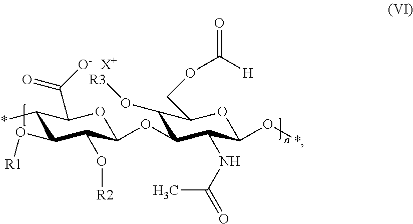
wherein
X is selected from H, Na, and K,
n stands for numbers from 100 to 10000, and
R1, R2, and R3 are selected independently of each other from —H, —C(O)—H, and

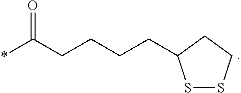 [SMILES: *C(=O)CCCCC1CCSS1]

8. The hair treatment agent according to claim 1, wherein the hair treatment agent includes, with respect to the weight thereof, 0.5 to 70 wt % of anionic and/or nonionic and/or cationic and/or amphoteric surfactant(s).

9. The hair treatment agent according to claim 1, wherein the hair treatment agent includes, with respect to the weight thereof, 0.05 to 7.5 wt % of at least one cationic polymer selected from the group consisting of
poly(methacryloyloxy ethyl trimethylammonium chloride) having an INCI name Polyquaternium-37,
quaternized cellulose derivatives having an INCI name Polyquaternium 10,
cationic alkyl polyglycosides,
cationized honey,
cationized guar derivatives,
polymeric dimethyldiallylammonium salts and copolymers thereof with esters and amides of acrylic acid and methacrylic acid,
copolymers of vinylpyrrolidone with quaternized derivatives of dialkylamino alkyl acrylate and methacrylate,
vinylpyrrolidone/vinylimidazolium methochloride copolymers,
quaternized polyvinyl alcohol,
Polyquaternium 2,
Polyquaternium-7,
Polyquaternium 17,
Polyquaternium 18,
Polyquaternium 24, and
Polyquaternium 27.

10. A method for improving at least one of the following properties:
tensile strength of keratin fibers, particularly human hair;
stabilization of the moisture balance of keratin fibers, particularly human hair;
combability of keratin fibers, particularly human hair;
slowing of the aging process of keratin fibers, particularly human hair;
ability of keratin fibers, particularly human hair, to be restructured during and after the permanent wave process;
reduction in the elasticity decrease of keratin fibers, particularly human hair, in the event of damage caused by atmospheric influences,
including:
applying to the keratin fibers a hair treatment agent, including, in each case with respect to the weight thereof,
a) 0.0001 to 10 wt % of at least one oligopeptide that comprises at least one amino acid sequence Glu-Glu-Glu 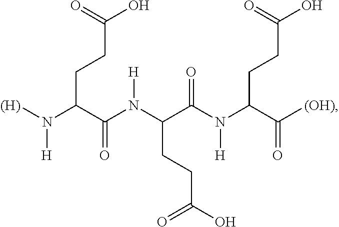
wherein the amino group can be free or protonated and the carboxyl groups can be free or deprotonated, and
b) 0.0001 to 10 wt % of at least one ester of hyaluronic acid with lipoic acid, formic acid, or mixtures thereof, and
leaving the hair treatment agent applied to the keratin fibers for 5 seconds to 10 minutes, and
rinsing the hair treatment agent out of the keratin fibers.
